(19) United States
(12) Patent Application Publication
(10) Pub. No.: US 2012/0191105 A1
(43) Pub. Date: Jul. 26, 2012

(54) DEVICE FOR EASILY INSTALLING AND DETACHING CONTACT LENSE FROM AN EYE

(76) Inventor: Syu-Guang Deng, Taipei Hsien (TW)

(21) Appl. No.: 13/010,784

(22) Filed: Jan. 21, 2011

(51) Int. Cl. A61F 9/00

(52) U.S. Cl. 606/107

(57) ABSTRACT

A device for easily installing and detaching contact lenses from an eye includes a contact lens attaching unit which is slightly larger than the area of an eye and has an concave surface for attaching with a contact eye; the surface having a highly coarse area; a retainer having a focus hole at a center region thereof for aligning to a center of the contact lens; a handle; an operating rod connecting to the attaching unit and the handle for pulling the attaching unit downwards so as to detach the contact lens from the eye; and a spring for restoring the contact lens to the original position. The device is used to align with an eye of the user. When the user pushes the operating rod, the attaching unit will interact so as to detach the contact lens from the eye.

### FIELD OF THE INVENTION

The present invention relates to the installation and detaching of contact lenses, in particular to a device for easily attaching and detaching a contact lens from an eye by that the eye can be protected from the infect of germs and the intrusions of dirt.

### BACKGROUND OF THE INVENTION

Currently, in detaching the contact lens, the user uses one hand to open the eye and then the user detaching the contact lens by two fingers. However, this operation will cause that the eyeball is hurt by the user's fingernail. Furthermore, currently, many people attaches some attaching sheets on the fingernail so as to provide the fingernail with beautiful colors and patterns, further the fingernail can be prolonged. However, the longer the fingernail, the easier the fingernail to be hurt. Further, the fingernail is hidden with plentiful of germs and dirt, if the contact lens is detached from the eye by using fingers directly, it is very easy that the germs or dirt will enter into the eye so as to infect the eye. However, the prior art way for detaching the contact lens from an eye is not a good one and thus it is necessary to be improved.

### SUMMARY OF THE INVENTION

The object of the present invention is to provide a device for easily attaching and detaching a contact lens from an eye by that a user can detach a contact lens, while the user's hand is unnecessary to touch the contact lens so as not to hurt the user's eye. In the operation of the present invention, the user's hand will not touch the eyeball of the user, and thus no germ will infect the user's eye.

To achieve above object, the present invention provides a device for easily attaching and detaching a contact lens from an eye, comprising: a contact lens attaching unit being a gal body with a size slightly greater than that of a pupil of an eye; one side of the contact lens attaching unit being concave and another opposite side thereof being convex; the concave side of the contact lens attaching unit being formed as a contact lens attaching surface; an arc of this side being matched to an arc of an eyeball; in operation, the contact lens attaching surface being attached to the contact lens installed on a surface of an eyeball for detaching the contact lens from the eyeball; at least one friction area being formed on the contact lens attaching surface; the friction coefficient of the friction area being higher than that of the other area of the contact lens attaching surface; the friction area causing that the contact lens attaching surface being easily attached to the contact lens attaching surface; a retainer being fixedly attached on the convex side of the contact lens attaching unit; one side of the retainer extending downwards; a center of the retainer being formed with a through hole; in use, the eye of the user can align to the through hole so as to assure that the contact lens attaching unit is exactly adhered to the pupil of the eye; a handle; the extending end of the retainer being attached to the handle; an operation rod being connected to the contact lens attaching unit and the handle; one end of the operation rod being connected to the contact lens attaching unit or the retainer; when the user assures that the contact lens attaching unit has aligned to the eyeball of the user, the operation rod can be pulled downwards so as to drive the operation rod downwards and thus the contact lens can be taken down; and a spring installed within the handle; one end of the spring being connected to the operation rod and another end of the spring being connected to an inner side of the handle; the spring providing a resilient force so that the contact lens attaching unit can return to the original position.

The various objects and advantages of the present invention will be more readily understood from the following detailed description when read in conjunction with the appended drawing.

### BRIEF DESCRIPTION OF THE DRAWINGS

FIG. 1 is an exploded view of the present invention.

FIG. 2 is a cross section view of the present invention.

FIG. 3 is a front view of the present invention.

FIG. 4 is a backside view of the present invention.

FIG. 5 is an assembly view showing the interior structure of the present invention.

FIG. 6 is a perspective view of the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

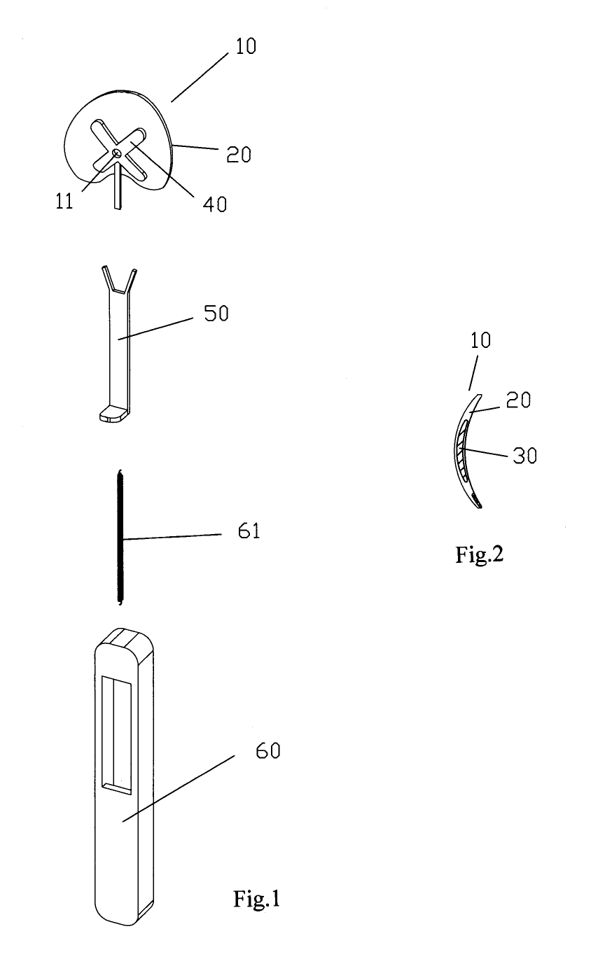

With reference to FIG. 1, the device for easily attaching and detaching a contact lens from an eye of the present invention is illustrated. The present invention includes the following elements.

A contact lens attaching unit 10 is a cambered gal body with a size slightly greater than that of a pupil of the eye. One side of the contact lens attaching unit 10 is concave and another opposite side thereof is convex. Preferably the gel body is transparent. An outer side of the gel body is formed with solid silicide and an interior thereof is filled with silica gel. The concave side of the contact lens attaching unit 10 is formed as a contact lens attaching surface 20. The arc of this side is matched to the arc of an eyeball. In operation, the contact lens attaching surface 20 is attached to the contact lens installed on the surface of an eyeball for detaching the contact lens from the eyeball. Referring to FIG. 2, it is illustrated that at least one friction area 30 is formed on the contact lens attaching surface 20. The friction coefficient of the friction area 30 is higher than that of the other area of the contact lens attaching surface 20. The friction area 30 causes that the contact lens attaching surface 20 can be easily attached to the contact lens attaching surface 20. With reference to FIG. 4, a backside view of the present invention is illustrated.

Besides, a lower side of the gal body can be formed with a notch for simplifying the detaching operation of the contact lens.

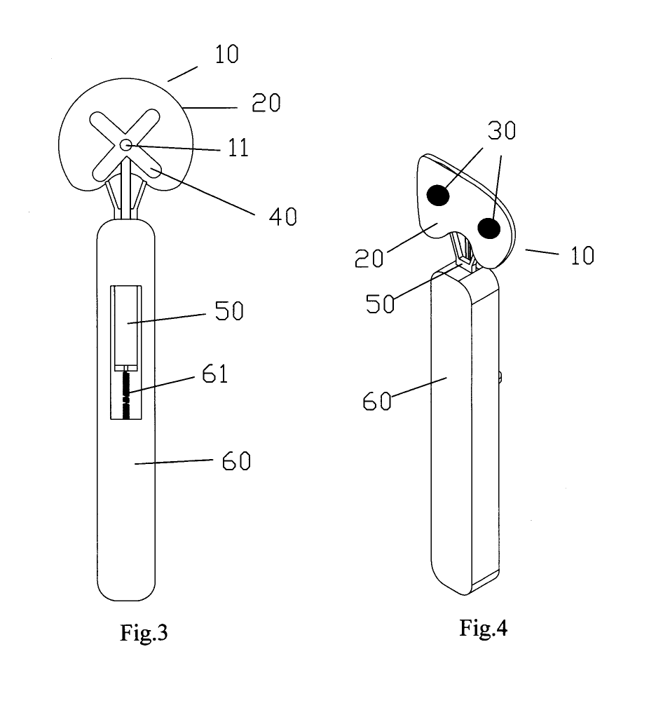

A retainer 40 is fixedly attached on the convex side of the contact lens attaching unit 10. One side of the retainer 40 extends downwards. Preferably, the retainer 40 has a part of cruciform. A center of the retainer 40 is formed with a through hole 11. In use, the eye of the user can align to the through hole so as to assure that the contact lens attaching unit 10 is exactly adhered to the pupil of the eye. Thus the contact lens can be taken down easily, as illustrated in FIG. 3.

A handle 60 serves to be held by the user and supports the contact lens attaching unit 10 and other elements of the present invention. The extending end of the retainer 40 is attached to the handle 60.

An operation rod 50 is connected to the contact lens attaching unit 10 and the handle 60. One end of the operation rod 50 is connected to the contact lens attaching unit 10 (or the retainer 40). Another end of the operation rod 50 protrudes out from an opening of the handle 60 so that the user can operate the operation rod 50. When the user assures that the contact lens attaching unit 10 has aligned to the eyeball of the user, the operation rod 50 can be pulled downwards so as to drive the operation rod 50 downwards and thus the contact lens can be taken down.

Preferably, the handle 60 and the operation rod 50 have curved outlooks so as to match the requirement of ergonometrics.

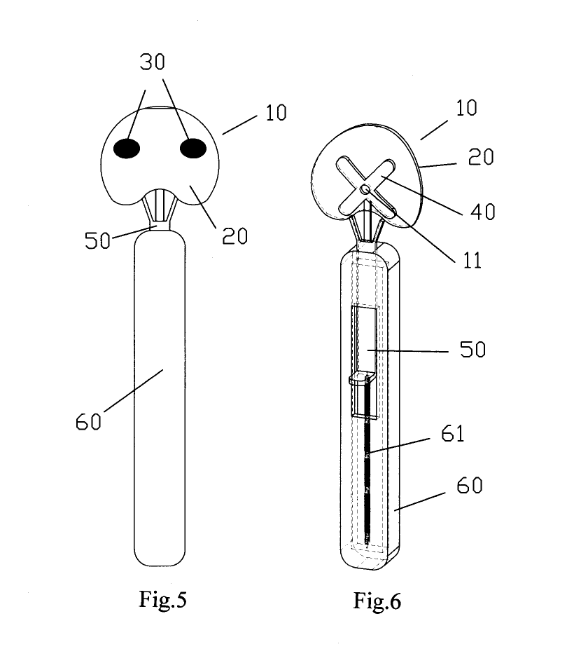

A spring 61 is installed within the handle 60. One end of the spring 60 is connected to the operation rod 50 (preferably, it is connected to a lower end of the operation rod 50) and another end of the spring 60 is connected to an inner side of the handle 60. In use of the present invention, when the operation rod 50 is pulled downwards, the contact lens attaching unit 10 is also pulled downwards and thus the contact lens can be taken down. When the operation rod 50 is released from the user, the spring 60 will cause the contact lens attaching unit 10 returns to the original position. Referring to FIG. 5, the assembly view of the present invention is illustrated.

In use of the present invention, firstly, the contact lens attaching surface 20 of the contact lens attaching unit 10 is attached to the contact lens and the through hole 11 of the retainer 40 is aligned to the eye center so that the user can assure that the contact lens attaching unit 10 almost covers the contact lens. Then the user pulls the operation rod 50 downwards so that the contact lens is also pulled down by the friction force of the friction area 30 of the contact lens attaching surface 20. When the user releases the operation rod 50, the retainer 40 and the contact lens attaching unit 10 will return to the original position by the resilient force of the spring 61.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A device for easily attaching and detaching a contact lens from an eye, comprising:
a contact lens attaching unit being a gal body with a size slightly greater than that of a pupil of an eye; one side of the contact lens attaching unit being concave and another opposite side thereof being convex; the concave side of the contact lens attaching unit being formed as a contact lens attaching surface; an arc of this side being matched to an arc of an eyeball; in operation, the contact lens attaching surface being attached to a contact lens installed on a surface of an eyeball for detaching the contact lens from the eyeball; at least one friction area being formed on the contact lens attaching surface; the friction coefficient of the friction area being higher than that of the other area of the contact lens attaching surface; the friction area causing that the contact lens attaching surface being easily attached to the contact lens attaching surface;
a retainer being fixedly attached on the convex side of the contact lens attaching unit; one side of the retainer extending downwards; a center of the retainer being formed with a through hole; in use, the eye of the user can align to the through hole so as to assure that the contact lens attaching unit is exactly adhered to the pupil of the eye;
a handle; the extending end of the retainer being attached to the handle;
an operation rod being connected to the contact lens attaching unit and the handle; one end of the operation rod being connected to the contact lens attaching unit or the retainer; when the user assures that the contact lens attaching unit has aligned to the eyeball of the user, the operation rod can be pulled downwards so as to drive the operation rod downwards and thus the contact lens can be taken down; and
a spring installed within the handle; one end of the spring being connected to the operation rod and another end of the spring being connected to an inner side of the handle; the spring providing a resilient force so that the contact lens attaching unit can return to the original position.

2. The device for easily attaching and detaching a contact lens from an eye as claimed in claim 1, wherein the gel body is transparent; an outer side of the gel body is formed with solid silicide and an interior thereof is filled with silica gel.

3. The device for easily attaching and detaching a contact lens from an eye as claimed in claim 1, wherein the retainer has a part of cruciform.

4. The device for easily attaching and detaching a contact lens from an eye as claimed in claim 1, wherein the handle and the operation rod have curved outlooks so as to match the requirement of ergonometric.

5. The device for easily attaching and detaching a contact lens from an eye as claimed in claim 1, wherein another end of the operation rod protrudes out of an opening of the handle so that the user can operate the operation rod.

6. The device for easily attaching and detaching a contact lens from an eye as claimed in claim 1, wherein a lower side of the gal body is formed with a notch for simplifying the detaching operation of the contact lens.
